(19) United States
(12) Patent Application Publication
(10) Pub. No.: US 2012/0191108 A1
(43) Pub. Date: Jul. 26, 2012

(54) ALL NATURAL VEGETABLE FIBER EXFOLIATING CLOTH

(76) Inventor: Gwen Vanessa Thomas, Miami, FL (US)

(21) Appl. No.: 13/353,996

(22) Filed: Jan. 19, 2012

Related U.S. Application Data
(63) Continuation-in-part of application No. 12/290,576, filed on Oct. 31, 2008, now abandoned.
(60) Provisional application No. 61/001,157, filed on Oct. 31, 2007.

(51) Int. Cl. A61B 17/50

(52) U.S. Cl. 606/131

(57) ABSTRACT

A washcloth for exfoliating the skin comprises a primary fabric panel and a secondary fabric panel, both of which may be made from vegetable fibers from the Malvaceae family. Both panels have a front side and a back side, and on each panel, the intersection of the front side with the back side defines at least a top peripheral edge, a bottom peripheral edge, a first side peripheral edge, and a second side peripheral edge. The panels are attached to each other along their bottom peripheral edges, and along a portion of the primary panel's side peripheral edges with the entirety of the secondary panel's side peripheral edges to form a pocket between the panels. A portion of the front side of the primary panel remains exposed to be used as a washcloth. A soap product is inserted and held within the pocket to be used in bathing.

This application is a continuation-in-part of non-provisional patent application Ser. No. 12/290,576 filed on Oct. 31, 2008, which is based on provisional patent application Ser. No. 61/001,157 filed on Oct. 31, 2007.

### BACKGROUND

Some embodiments may relate to a wash cloth for bathing and, more particularly, to an exfoliating wash cloth made of natural vegetable fibers.

Exfoliation is a common cosmetic technique that involves removing the dead skin cells on the outer surface of the skin (i.e., stratum corneum) to expose fresh living skin cells. Removing dead skin cells by exfoliating results in younger, brighter looking skin, as well as better penetration of active ingredients in skin care products (e.g., skin moisturizers). Exfoliating on a regular basis helps to generate new skin cells at a faster rate, thereby providing a healthier glow to the skin, as well as improved skin sensation.

The process of exfoliating the skin can be done by either a mechanical technique or with the use of chemical exfoliants. Mechanical exfoliants include micro-bead facial scrubs, natural granular substances, such as sugar or salt crystals, and abrasive clothes such as loofahs. Chemical exfoliants contain various acids and enzymes, including salicylic acid, glycolic acid, citric acid, and fruit enzymes.

While the above described mechanical and chemical exfoliants have proven to be effective in removing dead skin cells and improving skin appearance, the products containing these exfoliants can be relatively expensive compared to other traditional bathing products. Moreover, some people may be allergic to the rather exotic ingredients contained in both mechanical and chemical exfoliant products. Further, use of most mechanical and chemical exfoliants is limited to once or twice a week. A person using these exfoliants more often than recommended may be removing too many living skin cells and may increase the chance of skin irritation and damage.

Accordingly, there remains a desperate need in the cosmetic industry for an inexpensive, highly effective product for mechanically exfoliating the skin on a daily basis. More particularly, there remains a need for a wash cloth made of all natural vegetable fibers that is particularly effective in exfoliating the skin to remove dead skin cells and revitalize skin tone and skin sensation, and wherein the exfoliating wash cloth can be used on a daily basis without causing skin irritation or skin damage. There is a further need for an exfoliating wash cloth made up of all natural vegetable fibers, and wherein the wash cloth is structured to hold a soap product (e.g. bar soap, liquid soap or bath gel) while washing and exfoliating the skin using the wash cloth.

While certain aspects of conventional technologies have been discussed and presented to facilitate disclosure of some embodiments, Applicants in no way disclaim these technical aspects, and it is contemplated that the attached claims may encompass one or more of the conventional technical aspects discussed herein.

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is it known to be relevant to an attempt to solve any problem with which this specification is concerned.

### SUMMARY

Some embodiments may address one or more of the problems and deficiencies discussed above. However, it is contemplated that some embodiments may prove useful in addressing other problems and deficiencies in a number of technical areas. Therefore the claims should not necessarily be construed as limited to addressing any of the particular problems or deficiencies discussed herein.

Some embodiments may be directed to a wash cloth made of all natural vegetable fibers. The wash cloth may be particularly effective for exfoliating the skin to remove dead skin cells and revitalize skin tone and sensation. The exfoliating wash cloth may include a pocket for holding bar or liquid soap while rubbing the cloth against the skin when bathing.

An advantage provided by certain embodiments is that soap may be more effectively contained and distributed during bathing compared to traditional means of washing.

These and other advantages of some embodiments are more readily apparent with reference to the detailed description and accompanying drawings.

### BRIEF DESCRIPTION OF THE DRAWINGS

For a fuller understanding of the nature of some embodiments, reference should be made to the following detailed description taken in conjunction with the accompanying drawings in which:

FIG. 1 is a front perspective view of the primary panel of the all-natural vegetable fiber exfoliating cloth according to an embodiment.

FIG. 2 is a front perspective view of the secondary panel of the all-natural vegetable fiber exfoliating cloth according to an embodiment.

FIG. 3 is a front perspective view of the all-natural vegetable fiber exfoliating cloth according to the embodiments of FIGS. 1-2.

FIG. 4 a front perspective view of the all-natural vegetable fiber exfoliating cloth according to the embodiment of FIG. 3 further including a bar of soap being inserted into the pocket.

FIG. 5 is a side elevational view of the all-natural vegetable fiber exfoliating cloth according to the embodiment of FIG. 3 shown in cross-section taken along the line 5-5 in FIG. 3.

FIG. 6 is a side elevational view of the all-natural vegetable fiber exfoliating cloth according to the embodiment of FIG. 4 shown in cross-section taken along the line 6-6 in FIG. 4.

Like reference numerals refer to like parts throughout the several views of the drawings.

### DETAILED DESCRIPTION OF SOME EMBODIMENTS

The term “some” as used herein is defined as “none, or one, or more than one, or all.” Accordingly, the terms “none,” “one,” “more than one,” “more than one, but not all” or “all” would all fall under the definition of “some.” The term “some embodiments” may refer to no embodiments or to one embodiment or to several embodiments or to all embodiments. Accordingly, the term “some embodiments” is defined as meaning “no embodiment, or one embodiment, or more than one embodiment, or all embodiments.”

The terminology and structure employed herein is for describing, teaching and illuminating some embodiments and their specific features and elements and do not limit, restrict or reduce the spirit and scope of the claims or their equivalents.

More specifically, any terms used herein such as but not limited to “includes,” “comprises,” “has,” “consists,” and grammatical variants thereof do NOT specify an exact limitation or restriction and certainly do NOT exclude the possible addition of one or more features or elements, unless otherwise stated, and furthermore must NOT be taken to exclude the possible removal of one or more of the listed features and elements, unless otherwise stated with the limiting language “MUST comprise” or “NEEDS TO include.”

All references herein to any features or elements of some embodiments such as but not limited to “a,” “an,” “one,” “an additional,” “another,” or “the” feature or element, or grammatical variations thereof, unless otherwise specified by limiting language such as “ONLY ONE . . . ” wherein an exact number is specified along with the world “ONLY” or “LIMITED TO”, do NOT specifically prevent the inclusion or implementation of more than one of those features or elements, nor do they specifically admit that one or more of those features or elements must be used, unless otherwise specified by the limiting language “MUST have a” or “NEEDS to include one.”

All references herein including language such as but not limited to “one or more,” “at least one,” or grammatical variants thereof are nonlimiting and may be taken to mean that none of a certain feature or element may be used, or alternatively that only a singular feature or element may be used, or alternatively that only a plurality of elements or features may be used, or alternatively that either a singular or a plurality of features or elements may be used, or alternatively that none or one or more than one features or elements may be used. The use of this terminology herein does NOT specifically admit that more than one feature or element has to be used, nor does it specifically admit that only one feature or element has to be used, unless otherwise specified by the limiting language “MUST BE MORE THAN” or “CANNOT BE MORE THAN.” Therefore, whether or not a certain feature or element were limited to being used only once, either way it may still be referred to as “one or more features” or “one or more elements” or “at least one feature” or “at least one element.” Furthermore, the use of the terms “one or more” or “at least one” feature or element do NOT preclude there being none of that feature or element, unless otherwise specified by limiting language such as “there NEEDS to be one or more . . . ” or “one or more element is REQUIRED.”

Unless otherwise defined, all terms, and especially any technical and/or scientific terms, used herein may be taken to have the same meaning as commonly understood by one having an ordinary skill in the art.

Reference is made herein to some “embodiments.” It should be understood that an embodiment is an example of a possible implementation of any features and/or elements presented in the attached claims. Some embodiments have been described for the purpose of illuminating one or more of the potential ways in which the specific features and/or elements of the attached claims fulfill the requirements of uniqueness, utility and non-obviousness.

Use of the phrases and/or terms such as but not limited to “a first embodiment,” “a further embodiment,” “an alternate embodiment,” “one embodiment,” “an embodiment,” “multiple embodiments,” “some embodiments,” “other embodiments,” or variants thereof do NOT necessarily refer to the same embodiments. For example, language such as “some embodiments include a feature” followed by “some embodiments include an element” does NOT necessarily admit that both of the feature and the element are included in all of the same embodiments.

Unless otherwise specified, one or more particular features and/or elements described in connection with one or more embodiments may be found in one embodiment, or may be found in more than one embodiment, or may be found in all embodiments, or may be found in no embodiments.

Although one or more features and/or elements may be described herein in the context of only a single embodiment, or alternatively in the context of more than one embodiment, or further alternatively in the context of all embodiments, the features and/or elements may instead be provided separately or in any appropriate combination or not at all. Conversely, any features and/or elements described in the context of separate embodiments may alternatively be realized as existing together in the context of a single embodiment.

Any and all details set forth herein are used in the context of some embodiments and therefore should NOT be necessarily taken as limiting factors to the attached claims. Any descriptions of elements and/or features and/or the materials used to create those elements or features, or examples or methods included in the descriptions of the various embodiments are nonlimiting and are given as an illustration only. Accordingly, the embodiments can be manufactured, distributed, used, practiced, and carried out in numerous ways.

The attached claims and their legal equivalents can be realized in the context of embodiments other than the ones used as illustrative examples in the description herein.

Referring to the several views of the drawings, the all-natural vegetable fiber exfoliating cloth is shown in accordance with some embodiments. In each of these embodiments, the all-natural vegetable fiber exfoliating cloth is generally indicated as 10.

Any terminology included herein such as “exfoliating wash cloth” or “wash cloth” or “cloth” or “cloth 10” should be, unless otherwise specified, taken to be a reference to the all-natural vegetable fiber exfoliating cloth 10.

The exfoliating wash cloth 10 may be made up of all-natural vegetable fibers in order to be particularly effective for exfoliating skin to remove dead skin cells, thereby revitalizing skin tone and skin sensation. The fibers may be produced from plants in the genus Corchorus, family Malvaceae.

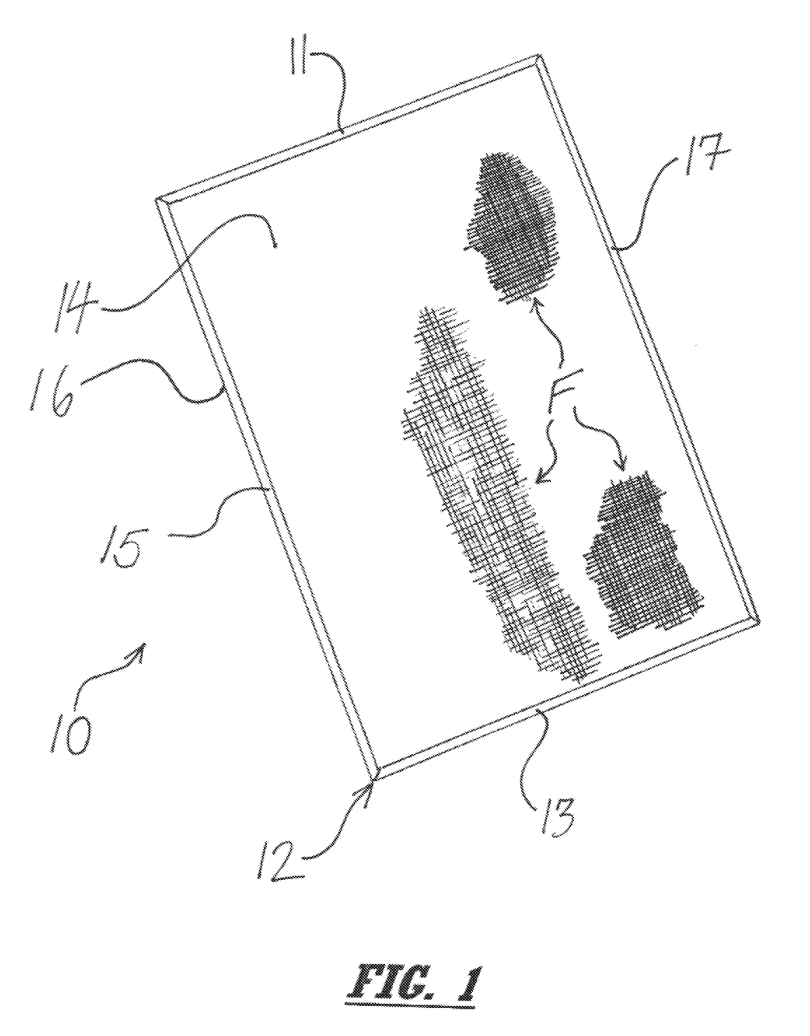

Reference is now made to FIG. 1. A primary panel 12 includes a front side 14 and a back side 16. The intersection of the front side 14 and the back side 16 define at least a top peripheral edge 11, a bottom peripheral edge 13, a first side peripheral edge 15 and a second side peripheral edge 17. The entire primary panel 12 may be made up of all-natural vegetable fibers F in order to be particularly effective for exfoliating skin to remove dead skin cells, thereby revitalizing skin tone and skin sensation. The fibers F may be produced from plants in the genus Corchorus, family Malvaceae. The primary panel 12 is shown as being substantially shaped as a rectangle; however it must be recognized that the functional aspects of the exfoliating cloth may be fully realized under a large variety of sizes and/or shapes of primary panels 12. An example of the size and shape of the primary panel 12 is a rectangular panel with peripheral edges ranging from 4 inches to 12 inches. Another example of the size and shape of the primary panel 12 is a square panel with all four peripheral edges having a length of 8 inches. These examples are non-limiting and serve only to illuminate a couple out of many possible sizes and shapes for the primary panel 12.

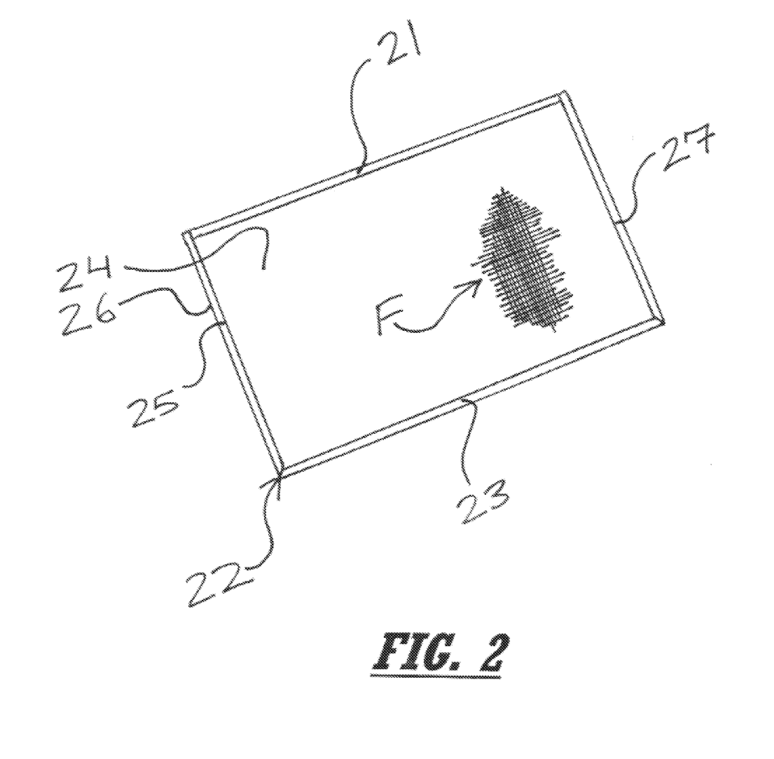

Additional reference is now made to FIG. 2. A secondary panel 22 includes a front side 24 and a back side 26. The intersection of the front side 24 and the back side 26 define at least a top peripheral edge 21, a bottom peripheral edge 23, a first side peripheral edge 25 and a second side peripheral edge 27. The entire secondary panel 22 may be made up of all-natural vegetable fibers F in order to be particularly effective for exfoliating skin to remove dead skin cells, thereby revitalizing skin tone and skin sensation. The fibers F may be produced from plants in the genus Corchorus, family Malvaceae. The secondary panel 22 is shown as being substantially shaped as a rectangle; however it must be recognized that the functional aspects of the exfoliating cloth may be fully realized under a large variety of sizes and/or shapes of secondary panels 22.

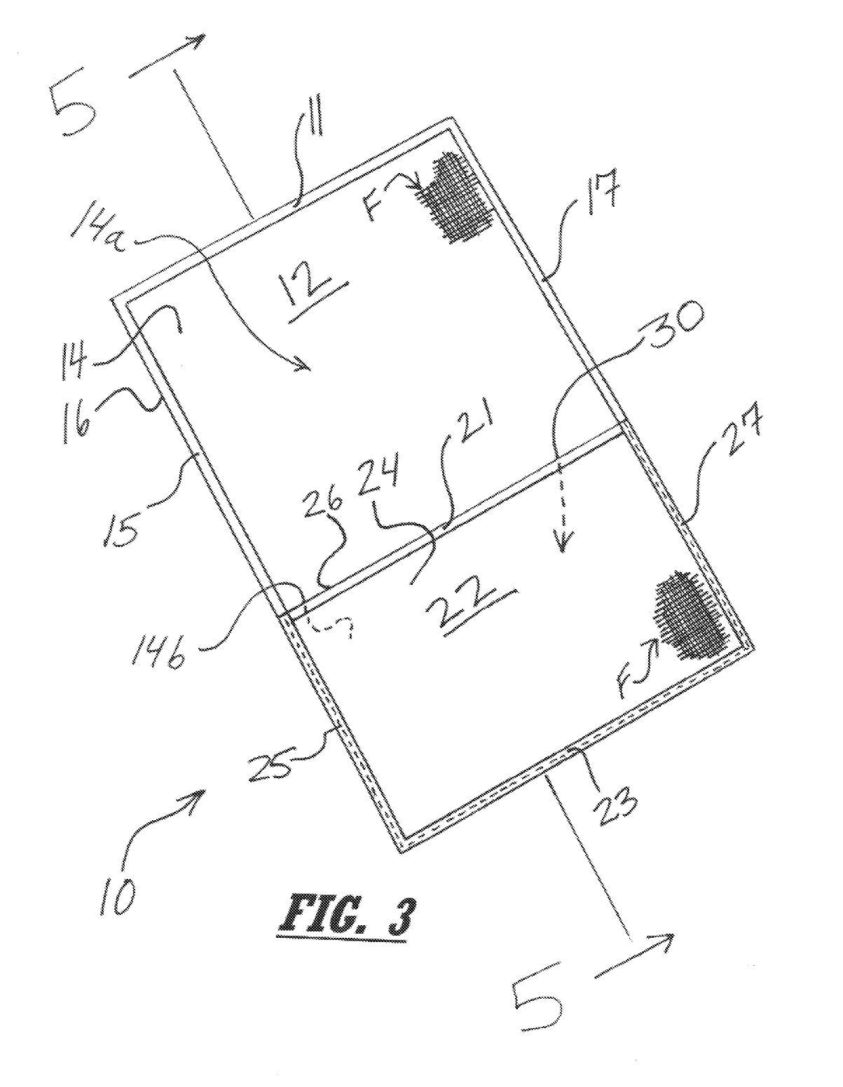

Additional reference is now made to FIG. 3. The secondary panel 22 is stitched or otherwise attached to the primary panel 12 in order to form a pocket 30. More specifically, the entirety of the bottom peripheral edge 23 is stitched or otherwise attached to the entirety of the bottom peripheral edge 13, while the entirety of the first side peripheral edge 25 is attached to only a portion and not the entirety of the first side peripheral edge 15, and similarly the entirety of the second side peripheral edge 27 is attached to only a portion and not the entirety of the second side peripheral edge 17. The top peripheral edge 21 is left completely detached from the primary panel 12, thereby serving as an opening to the pocket 30. The top peripheral edge 11 may or may not attach to the secondary panel 22 in any way at all. The formation of the pocket 30 divides the front side 14 of the primary panel 12 into two distinctive areas: an exposed portion 14a and a covered portion 14b. The exposed portion 14a represents a portion of the exfoliating cloth 10 that may be used as an independent washcloth i.e. without the interference of any soap or from the other panel 22. This washcloth portion 14a is very important to the structural and functional identity of the washcloth 10, as without it, washing would be much less convenient. The adjacent proximity of the washcloth portion 14a to the pocket 30 allows the washcloth portion 14a to always be substantially soaked in both soap and water (i.e. because the soap will be within the pocket 30) without actually needing to be in the way of the soap or the other panel 22. In order to preserve the functional integrity of the cloth 10, it is important that the washcloth portion 14a be substantially large enough to be used in contact with the body (i.e. similar to how a normal wash cloth is used). Therefore, the washcloth portion 14a should make up a substantial percentage of the total front side surface area 14. In one embodiment, the exposed area 14a should be at least 50% of the front side area 14. In other embodiments, it is possible that 14a may make up more or less than 50% of the front side area 14.

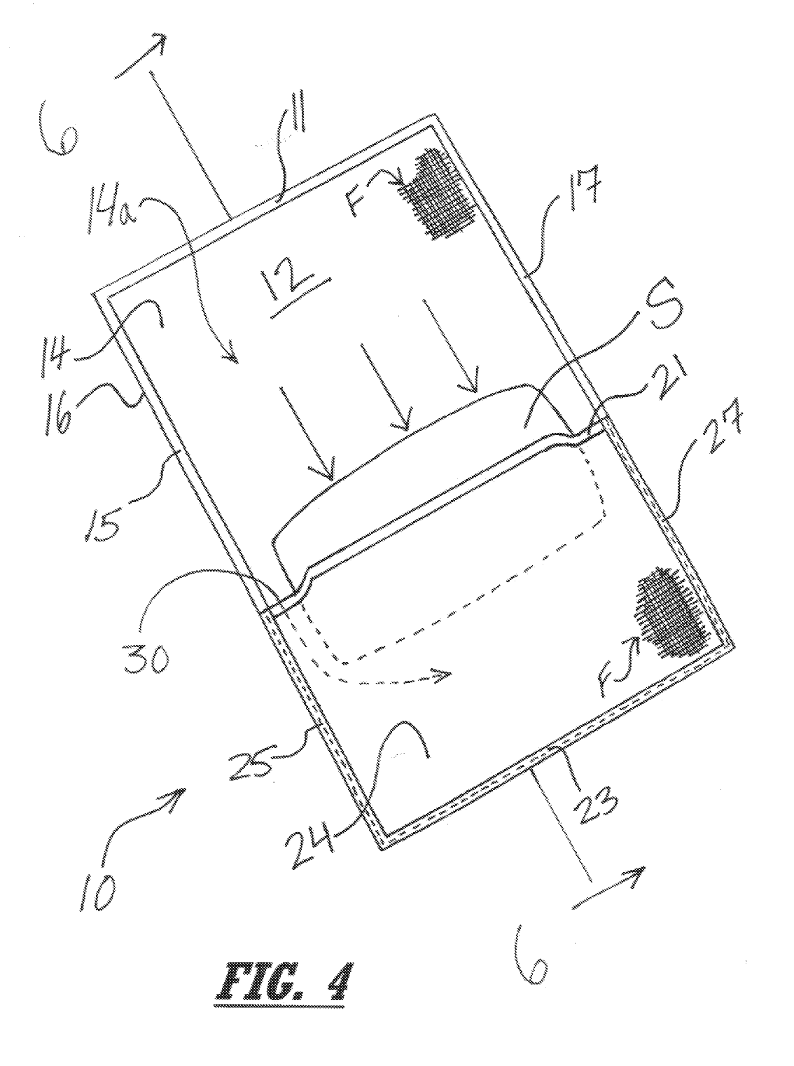

Additional reference is now made to FIG. 4. A bar of soap S can be seen being inserted into the pocket 30. Once the bar of soap S is within the pocket 30, the entire cloth 10 may be soaked and used for washing. More specifically, once water enters the pocket 30 and begins mixing with the bar of soap S, both panels 12 and 22 will start to become soaked with soapy suds. The washcloth portion 14a will be particularly effective for washing the body at this point. As shown in FIG. 4, the soap S may be a bar of soap S. Alternatively, the soap S may be a liquid or bath gel, or any other known washing products.

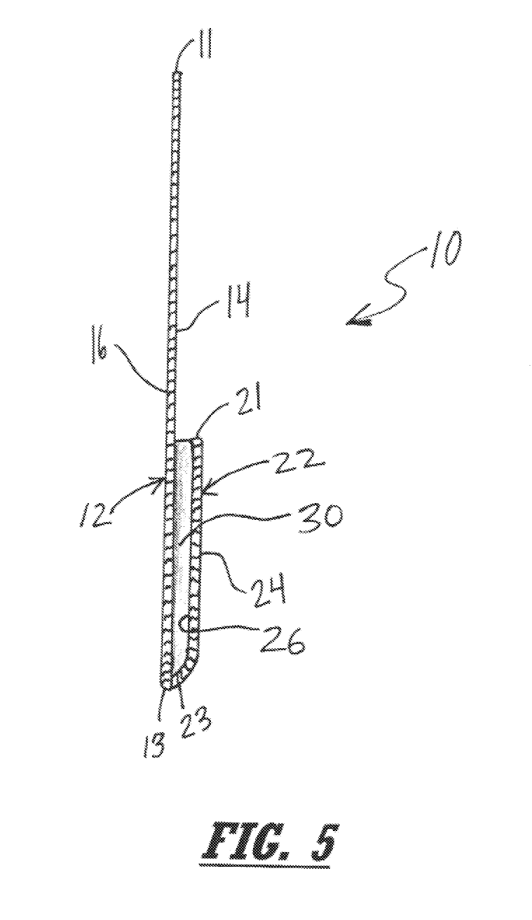

Additional reference is now made to FIG. 5. A cross-sectional view of FIG. 3 fully illuminates how the pocket 30 is formed between the panels 12 and 22.

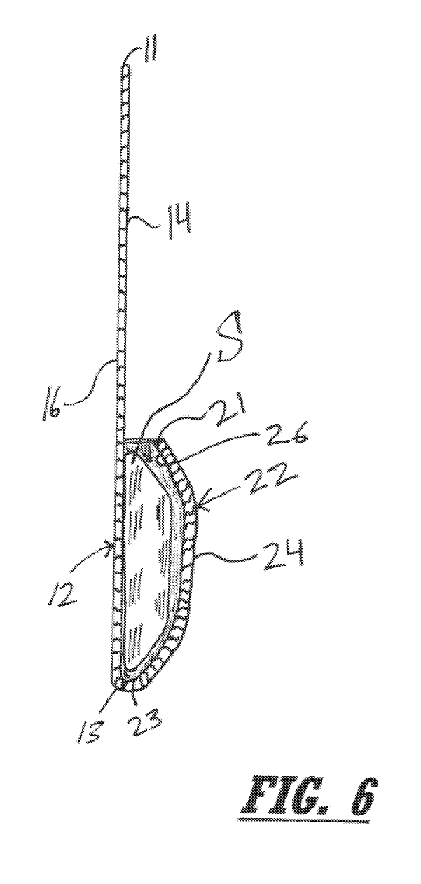

Additional reference is now made to FIG. 6. A cross-sectional view of FIG. 4 fully illuminates how the soap S fills the pocket 30.

Throughout the detailed description and the accompanying drawings enclosed herein, some embodiments have been shown, described and detailed, wherein a variety of possible elements and/or features may be formed and configured in different ways. Accordingly, any and all possible combinations of the elements and/or features described in accordance with these various embodiments may be desirable to manufacturers and/or may help to more successfully meet customers' specific needs and/or preferences. Consequently, any and all possible combinations of the features or elements of one embodiment or more than one embodiment or all embodiments mentioned herein are fully considered within the spirit and scope of the attached claims and their legal equivalents.

Thus, some embodiments of an all-natural vegetable fiber exfoliating cloth have been disclosed. Other embodiments are contemplated and envisioned, and therefore it is recognized that departures from the embodiments described in this disclosure may certainly exist within the spirit and scope of the attached claims and their legal equivalents. Those having an ordinary skill in the will envision other possible variations and modifications to features and/or elements of the embodiments, and they will envision other possible embodiments, all of which may fall within the spirit and scope of the attached claims. The spirit and scope of the attached claims is therefore NOT limited by the descriptions and illuminations of the embodiments that have already been presented, but rather the spirit and scope can only be defined by the attached claims and their legal equivalents as interpreted under the doctrine of equivalents. Variations, alternatives, adjustments, modifications, tunings, and deviations from the embodiments of the instant disclosure are fully contemplated and envisioned within the spirit and scope of the attached claims.

What is claimed is:

1. A washcloth for exfoliating the skin comprising:
a primary panel formed of woven vegetable fibers derived from the Malvaceae family of plants, said primary panel including a front side and a back side, wherein the intersection of the front side and the back side defines at least a top peripheral edge, a bottom peripheral edge, a first side peripheral edge, and a second side peripheral edge;
at least one secondary panel formed of said woven vegetable fibers and including a first side and a back side, wherein the intersection of the front side and the back side defines at least a top peripheral edge, a bottom peripheral edge, a first side peripheral edge, and a second side peripheral edge; and
wherein said at least one secondary panel is positioned and disposed in overlying relation to said primary panel with said back side of said secondary panel in opposing relation to a portion of said front side of said primary panel thereby forming a pocket between the primary panel and the secondary panel, and wherein the entire bottom peripheral edge of said secondary panel is attached to the entire bottom peripheral edge of said primary panel, and wherein the entire first side peripheral edge of said secondary panel is attached to only a portion and less than the entirety of the first side peripheral edge of said primary panel, and wherein the entire second side peripheral edge of said secondary panel is attached to only a portion and less than the entirety of the second side peripheral edge of said primary panel, and wherein the top peripheral edge of said secondary panel extends from the first side peripheral edge to the second side peripheral edge of said primary panel, such that an exposed portion of the front side of said primary panel remains exposed and not covered by said secondary panel, and wherein the top peripheral edge of said secondary panel remains separated from said primary panel thereby defining a pocket opening communicating with an interior of said pocket between said primary and secondary panels, and wherein the top peripheral edge of said primary panel remains entirely separated from said secondary panel.

2. The washcloth as recited in claim 1 wherein said exposed portion of the front side of said primary panel makes up at least 50% of the total area of the entire front side of said primary panel.
